(19) United States
(12) Patent Application Publication
(10) Pub. No.: US 2023/0165523 A1
(43) Pub. Date: Jun. 1, 2023

(54) METHODS AND SYSTEMS FOR MEASURING STRENGTHS AND VARIATIONS OF YING-NUTRIENT QI AND WEI-DEFENSIVE QI

(71) Applicant: Qiang WEI, Zhengzhou (CN)

(72) Inventor: Qiang WEI, Zhengzhou (CN)

(21) Appl. No.: 18/154,031

(22) Filed: Jan. 12, 2023

Related U.S. Application Data
(63) Continuation-in-part of application No. PCT/CN2021/106121, filed on Jul. 13, 2021.

(30) Foreign Application Priority Data
Jul. 14, 2020 (CN) 202010672275.5

(51) Int. Cl. A61B 5/00; A61B 5/01

(52) U.S. Cl. CPC A61B 5/4854; A61B 5/015; A61B 5/0008; A61B 5/6843; A61B 5/6826; A61B 5/6838; A61B 2562/0219

(57) ABSTRACT

A method for measuring the strength and variation of Ying-nutrient Qi: heating tissue to a set temperature on a site near a human meridian, and then measuring infrared radiation to calculate an obscuration ratio so that a numerical representation of the strength of Ying-nutrient Qi can be obtained. A Ying-nutrient Qi measurement system, including at least two pairs of infrared temperature sensors and contact temperature sensors, a CPU, and a storage device, wherein the contact temperature sensors is in direct contact with a human body, and every infrared temperature sensor is paired with a contact temperature sensor to measure the temperature of a site. By measurement, whether a patient has blood stasis or phlegm retention can be inferred. In addition, a fluctuation condition of Ying-nutrient Qi or the relative strength of Ying-nutrient Qi/Wei-defensive Qi of different meridians can further provide auxiliary bases for syndrome differentiation of six meridians.

### BACKGROUND

The present application relates to the field of traditional Chinese medicine, and in particular to methods and systems for measuring strengths and variations of Ying-nutrient Qi and Wei-defensive Qi in meridians by measuring infrared thermal radiation and body surface temperature.

Over the years, people have conducted extensive and in-depth research on meridians including research on meridians themselves and using meridian information to help with diagnosis. The methods used include the resistance/conductivity method, high-frequency vibroacoustic method, spectroscopy method, laser intensity method, isotope tracer method and so on.

Meridian research using body surface temperature or infrared thermal imaging cameras is another important branch, and many achievements have been obtained so far. For example, Katsusuke Serizawa and Yu Fenglan found that the temperature of acupoints is generally higher than that of non-acupoints. Xu Jinsen, Hu Xianglong et al. obtained the high-temperature lines along the meridians by using infrared thermography after heating the acupoints and proved the existence of meridians from a new perspective. Zhang Dong, Liu Ruiting, and others found that piercing Hegu would cause the temperature of Yingxiang point to rise. In contrast, Ma Huimin and others found that after piercing Quchi, the infrared temperature of Quchi, Wenliu, and Shousanli would fall.

Another approach is to measure the temperature of acupoints to measure the strength of meridian Qi/blood or to know whether the person is sick (help with diagnosis). Ogihara found that the patient will feel uncomfortable if the temperature difference between the left and right of the same acupoint exceeds 0.5 degrees. Zhao Rongzhu, Gao Yanbin, etc., found that the temperature difference between the left and right acupoints of gastritis and diabetic patients is higher than that of healthy people. Experiments by Li Zishuang and Lin Huilan found that the temperature in Ganshu and Taichong in patients with liver disease is higher than that in healthy people. At present, the main idea of auxiliary diagnosis is to use thermal resistance or thermocouple to measure the skin surface temperature or to use an infrared thermal imager to measure the temperature and then compare the temperature of the same acupoint on the left and right sides. If the temperature difference is too large (such as exceeding 0.5 degrees), there is a problem with the corresponding meridians/viscera.

### SUMMARY

A method for measuring the strength of Ying-nutrient Qi, comprising: selecting a site on a running path of a meridian in a human body, heating the surrounding tissues to a set temperature, and then measuring infrared thermal radiation at the site to calculate an obscuration ratio, thereby obtaining a numeralized representation of the strength of the Ying-nutrient Qi.

According to an embodiment of the present application, furthermore: measuring the strengths of the Ying-nutrient Qi in multiple meridians in a human body at the same time and then comparing the multiple results obtained to obtain the relative strength of the Ying-nutrient Qi among different meridians.

A system for measuring the strength and variation of Ying-nutrient Qi in a human body, comprising: at least two pairs of infrared temperature sensors and contact temperature sensors, a CPU, and a storage device, wherein the contact temperature sensors is in direct contact with a human body, and every infrared temperature sensor is paired with a contact temperature sensor to measure the temperature of a site.

According to an embodiment of the present application, furthermore, the storage device stores two types of temperature data and corresponding timestamps at the same time, with a storage duration of greater than 30 minutes.

According to an embodiment of the present application, furthermore, the system also includes a Bluetooth or WIFI communication module for communicating with a smartphone.

According to an embodiment of the present application, furthermore, the storage device is a storage device of a smartphone.

According to an embodiment of the present application, the system also includes pressure sensors for measuring the pressure of temperature sensors on the skin.

According to an embodiment of the present application, furthermore, the system also includes a mechanism capable of precisely controlling the pressure of the temperature sensors on the skin.

According to an embodiment of the present application, furthermore, the system also includes one or more gyroscopes for detecting subject motion.

According to an embodiment of the present application, the system also includes clips capable of fixing the temperature sensors on a side of fingers.

According to an embodiment of the present application, furthermore, the system also includes pressure sensors for measuring the pressure of the temperature sensors on the skin.

A system for measuring the strength and variation of Ying-nutrient Qi in a human body, comprising: at least two pairs of infrared radiation sensors and contact temperature sensors, a CPU, and a storage device, wherein the contact temperature sensors are in direct contact with a human body, and every infrared radiation sensor is paired with a contact temperature sensor to measure a site.

According to an embodiment of the present application, the storage device stores two types of data and corresponding timestamps at the same time, with a storage duration of greater than 30 minutes.

According to an embodiment of the present application, the system also includes a Bluetooth or WIFI communication module for communicating with a smartphone.

According to an embodiment of the present application, furthermore, the storage device is a storage device of a smartphone.

According to an embodiment of the present application, the system also includes pressure sensors for measuring the pressure of temperature sensors on the skin.

According to an embodiment of the present application, the system also includes one or more gyroscopes for detecting subject motion.

According to an embodiment of the present application, the system also includes clips capable of fixing the temperature sensors on a side of fingers.

A system for measuring the strength of Wei-defensive Qi, comprising: a CPU, a storage device, at least two to-be-heated mediums capable of contacting and being heated by a human body, and corresponding temperature sensors placed within the mediums, wherein the CPU records a temperature-rising curve of the to-be-heated medium, and calculates an indicator such as a temperature-rising rate for evaluating the strength of the Wei-defensive Qi at the corresponding site.

According to an embodiment of the present application, furthermore, the to-be-heated medium is covered with a thermal insulating layer.

### BRIEF DESCRIPTION OF THE DRAWINGS

In order to more clearly illustrate the embodiments of the present application, the following will briefly introduce the accompanying drawings that need to be used in the description of the embodiments.

FIG. 1 is an oscillogram obtained by a system for measuring Ying-nutrient Qi according to the present application, showing changes in body surface temperature and no change in infrared radiation;

FIG. 2 is an oscillogram obtained by a system for measuring Ying-nutrient Qi according to the present application, showing abnormality (burrs) caused by Qi-blood stasis in meridians;

FIG. 3 is an oscillogram obtained by a system for measuring Ying-nutrient Qi according to the present application, showing abnormality (reversal) caused by stagnation in meridians;

FIG. 4 is diagram of a human body surface hierarchical model for understanding the present application;

FIG. 5 is a flow chart of a method for measuring the strength of Ying-nutrient Qi;

FIG. 6 is a structural diagram of a to-be-heated medium and the accessory structure used in a method or system for measuring Wei-defensive Qi;

FIG. 7 is a schematic diagram of a temperature-rising curve obtained by using a method for measuring Wei-defensive Qi according to the present application.

FIG. 8 is a structural diagram of a minimum system for measuring Ying-nutrient Qi according to the present application;

FIG. 9 is a structural diagram of a system for measuring Ying-nutrient Qi according to the present application;

FIG. 10 is a structural diagram showing how to use a pressure sensor to measure the pressure of a temperature sensor on the skin.

FIG. 11 is a layout diagram of a composite structure that includes a pressure sensor, a temperature sensor, and a pressure adjustment actuator.

FIG. 12 is a layout diagram of a composite structure that includes a pressure sensor, a temperature sensor, and a clip.

FIG. 13 is a structural diagram of a system for measuring Wei-defensive Qi according to the present application;

### DETAILED DESCRIPTION OF THE EMBODIMENTS

The following will clearly and completely describe the embodiments of the present application with reference to the accompanying drawings. Obviously, the described embodiments are only some, not all, embodiments of the present application. Based on the embodiments of the present application, all other embodiments obtained by persons of ordinary skill in the art without making creative efforts belong to the protection scope of the present application.

Before explaining the specific implementation, three basic principles used in the present application are firstly explained: First, the Qi running inside and outside the meridian includes Ying-nutrient Qi and Wei-defensive Qi. That is to say, the channel in “the Ying-nutrient Qi runs inside the channel, and Wei-defensive Qi outside the channel” are meridians rather than blood vessels. Second, there will be a small amount of seeping while the Ying-nutrient Qi runs in meridians. The more prosperous the Ying-nutrient Qi, the more seepage (but even more seeping out when the meridians are blocked). When the Ying-nutrient Qi seeps out, it will generate heat, and the product will affect (shade) the infrared radiation. Third, the Wei-defensive Qi outside the meridians has the function of keeping warm. If the Wei-defensive Qi is strong, the heat dissipation through convection and conduction will be slow, and the temperature of the corresponding parts of a human body (especially the internal temperature) will be higher. However, the Wei-defensive Qi basically does not affect the transmission of infrared rays.

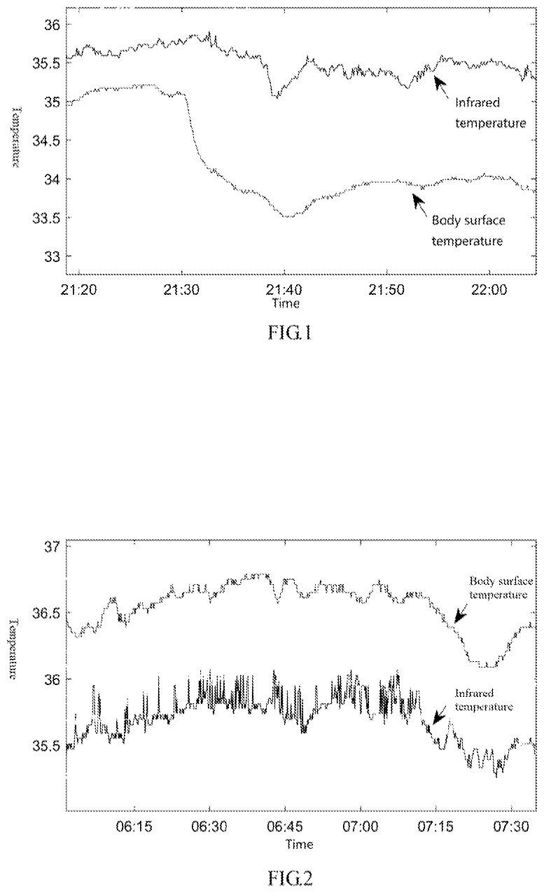

The following analysis (materials) are provided for the establishment of these three principles: First, according to an implementation method of the present application, when measured in a human body(especially at the beginning of traditional twelve two-hour periods), there will often be body surface temperature changes, but the infrared radiation remains constant at the time (FIG. 1). Wei-defensive Qi can well explain this phenomenon. That is, the heat production and internal temperature of the human body remain unchanged at this time, and Wei-defensive Qi does not block infrared rays, so the infrared radiation remains unchanged. However, the change of Wei-defensive Qi leads to the change of heat dissipation rate, so the body surface temperature changes accordingly.

Secondly, the waveform in FIG. 2 can often be seen for patients with blood stasis or phlegm-fluid retention—the infrared radiation presents sharp changes (burrs). However, the patient's body surface temperature does not change much. When measuring on hands, it often happens that waveforms measured on some fingers have burrs, but waveforms measured on adjacent fingers have not, which can rule out changes in infrared radiation caused by changes in blood flow velocity. This waveform cannot be explained by Wei-defensive Qi but can only be explained by introducing the mechanism that Ying-nutrient Qi generates heat. That is to say, for these patients, the flow of Ying-nutrient Qi is no longer smooth but intermittent, and the Ying-nutrient Qi also leaks intermittently to generate heat, and a sudden change in infrared radiation will appear. The explanation for the heat generated by Ying-nutrient Qi seepage is also consistent with the current research results that the temperature along the meridians is higher than that around the meridians.
Thirdly, according to the explanation of Ying-nutrient Qi seeping generate heat, the temperature on the upstream part of the meridian (for example, Hegu is on the upstream part when needling in Quchi) should rise when needling (because the flow of Ying-nutrient Qi is blocked). However, existing studies have shown that infrared radiation (infrared temperature) on the upstream part of the meridian (against the running direction of the Ying-nutrient Qi) will decrease during acupuncture. Therefore, the most reasonable explanation is that seeping and heating products of Ying-nutrient Qi will block infrared radiation.
The test carried out according to the implementation method of the present application also shows that when the temperature of finger tissue is fixed at the temperature of the constant temperature water tank, the infrared radiation of healthy people with strong Qi is lower when compared with people with weak Qi (the difference between infrared radiation temperature and the temperature of the constant temperature water tank is larger). This phenomenon also confirms that Ying-nutrient Qi seeps out and shields infrared radiation.
Finally, if one agrees with the above inference, one must also agree that Ying-nutrient Qi and Wei-defensive Qi are running inside and outside the meridians simultaneously, and the channels in “the Ying-nutrient Qi runs inside the channel, and Wei-defensive Qi outside the channel” are meridians rather than blood vessels.

According to the principles mentioned above, the meridian model shown in FIG. 4 can be constructed. The model includes the inner body layer, meridian layer, Ying-nutrient Qi seepage layer, Wei-defensive Qi layer, and epidermis (Ying-nutrient Qi seepage layer and Wei-defensive Qi layer are actually integrated rather than distinct). In the model, the inner layer is the heat source, and the heat in the limbs mainly comes from tissues such as blood and muscles (but also from the seepage layer of Ying-nutrient Qi). The meridian layer is a thin line with a diameter of one millimeter or several millimeters, corresponding to the meridians of a human body. The seepage layer will generate heat and increase the temperature of the inner layer, and at the same time, block the infrared rays emitted by the inner layer (of course, this layer itself also emits infrared rays, but its strength is relatively small, so the proportion of these infrared rays that are blocked can be ignored). The Wei-defensive Qi layer plays a role in keeping warm. The stronger the Wei-defensive Qi is, the less heat will be lost through convection (that is the embodiment of the function of the Wei-defensive Qi keeping the body warm), and the corresponding inner layer temperature and body surface temperature will be higher. However, the Wei-defensive Qi layer does not affect the transmission of infrared radiation.

It should be noted that this model is an equivalent model. That is to say, the model is mainly given for easy understanding of subsequent embodiments, and its structure is entirely different from the physiological skin model. Therefore, a layer in this model cannot and should not be equated with a structure in any human tissue.

The methods and systems of the present application are provided below according to the foregoing principles and models.

A method for measuring the strength of Ying-nutrient Qi, comprising: selecting a site on a running path of a meridian in a human body, heating the surrounding tissues to a set temperature, and then measuring infrared thermal radiation at the site to calculate an obscuration ratio, thereby obtaining a numeralized representation of the strength of the Ying-nutrient Qi.

During implementations, heat sources such as infrared lamps cannot be used for heating, otherwise, uneven heating will be caused at different sites in a human body. A recommended method includes immersing hands in a thermostatic water tank. A recommended temperature is 38° C., which is higher than the temperature of the hands and does not lead to sweating which affects the ratio at which infrared rays are obscured. After a temperature field at different sites on hands becomes even by being heated for a period of time (such as 15 minutes), a miniature infrared temperature sensor can be placed on a running path of a meridian in each finger to measure infrared radiation. It can be found from the measurement results that the infrared temperature got by different sensor is different, and is generally lower than 38° C. Here, the following formula may be used to obtain an obscuration ratio:

k=(38−To)/38=1−To/38 (1)

k obtained here is a value between 0 and 1, and the larger the value, the stronger the strength of the corresponding Ying-nutrient Qi. Nevertheless, for the sake of simplicity, a temperature difference may also be used directly to evaluate the strength of the Ying -nutrient Qi.

ΔTo=38−To (2)

The larger the temperature difference obtained, the stronger the strength of the corresponding Ying-nutrient Qi is. This is also the practice to be used hereinafter. That is, the temperature difference value is used to represent the strength of Qi & blood.

In addition to measurement on fingers, the aforementioned method can also be applied to other parts of limbs. Here, the thermostatic water tank can be changed to other thermostatic heating means capable of covering the sites to be measured. However, this method cannot be applied to the trunk, since it is difficult to ensure constant internal temperature due to many other internal heat sources (internal organs, etc.).

According to an embodiment of the present application, furthermore, the infrared temperature sensors each have a waterproof structure.

There are a variety of measures for achieving the waterproof structure. For example, the housing of each sensor can be sealed with adhesive or a rubber seal ring, etc. Due to the use of the waterproof structure, the infrared temperature sensors can be immersed in water for use. As such, a certain site on a human body, together with the infrared temperature sensor, can be immersed in the thermostatic water tank; after a period of time, the infrared radiation temperature of a site where a meridian passes nearby is measured in the water tank (without taking the measured site out of the water tank); and then, an obscuration coefficient of the seepage of Ying-nutrient Qi can be obtained by using formula (1), whereby the strength of the Ying-nutrient Qi can be evaluated.

Such a practice of measuring the intensity of infrared radiation after heating an object to a preset temperature is seemingly somewhat similar to measuring emissivity. But the two should not be confused. This is because the emissivity is only applicable to an object made of a single material (such as metal, plastic, cement, etc.), while a human body is a complex organism in which infrared radiation is affected by many factors such as the blood flow rate, the strengths of Ying-nutrient Qi and Wei-defensive Qi and sweating. Therefore, the concept of emissivity should not be used here, nor can infrared radiation and body surface temperature be expected to achieve a fixed ratio.

It should be noted that there is an alternative method to achieve the object of this embodiment. For example, in case the infrared temperature sensor may not have a strict waterproof design, a tester can stick the sensor to a finger, then wear a waterproof glove, and immerse the hand into the thermostatic water tank. Such an alternative design is essentially the same as this embodiment, except in that the waterproof structure is expanded to include the hands of a test subject and the sensors.

According to an embodiment of the present application, furthermore: measuring the strengths of the Ying-nutrient Qi in multiple meridians in a human body at the same time and then comparing the multiple results obtained to obtain the relative strength of the Ying-nutrient Qi among different meridians.

During simultaneous measurement and comparison, measuring multiple meridians in multiple fingers is also the most convenient way. The reason lies in that the fingers are anatomically similar, showing good comparability. After the relative strength is obtained, it can be used as intermediate information to assist diagnosis. In fact, a traditional Chinese medicine doctors often makes a prescription based on the relative strength of Ying-nutrient Qi, instead of the absolute strength of the Ying-nutrient Qi (patients with longstanding disease may have weaker Ying-nutrient Qi in all meridians than normal people). For example, if the Ying-nutrient Qi corresponding the liver and kidneys is weaker, targeted treatment can be carried out. However, the strength of Qi and blood in the meridians should not be directly associated here with the name of a disease or a syndrome. The connection must be made by a doctor by using various other symptoms as well.

The thermostatic method as mentioned above cannot be used for sites on the trunk and the like, and an alternative method can be used.

A method for measuring the strength of Ying-nutrient Qi, comprising: measuring a temperature at a certain depth under the skin with a penetrating temperature sensor near a running path of a meridian in a human body, and then measuring infrared thermal radiation at the place to calculate an obscuration ratio, thereby obtaining a numeralized representation of the strength of the Ying-nutrient Qi.

According to an embodiment of the present application, a temperature measured deep beneath the human skin by a penetrating temperature sensor (for example, a thermal resistor may be installed at the tip of an acupuncture needle) can be used to replace the constant temperature of 38° C. in the formula (1), and the infrared temperature measured at the same site is then brought into the formula, whereby the obscuration coefficient k of a seepage layer of Ying-nutrient Qi may also be obtained. During implementation, it should be noted that a penetration position should keep away from but approach the meridians as much as possible. The reason for this is that the seepage of Ying-nutrient Qi is not limited to regions directly above the meridians, but spreads nearby. Therefore, a near-region measurement may have a similar effect. At the same time, keeping away from the meridians can prevent damage caused by blocking the Qi-blood circulation. However, this method is invasive and may also disrupt the Qi-blood circulation and cause possible damage if the meridians are pierced accidentally. Therefore, this is not a recommended practice.

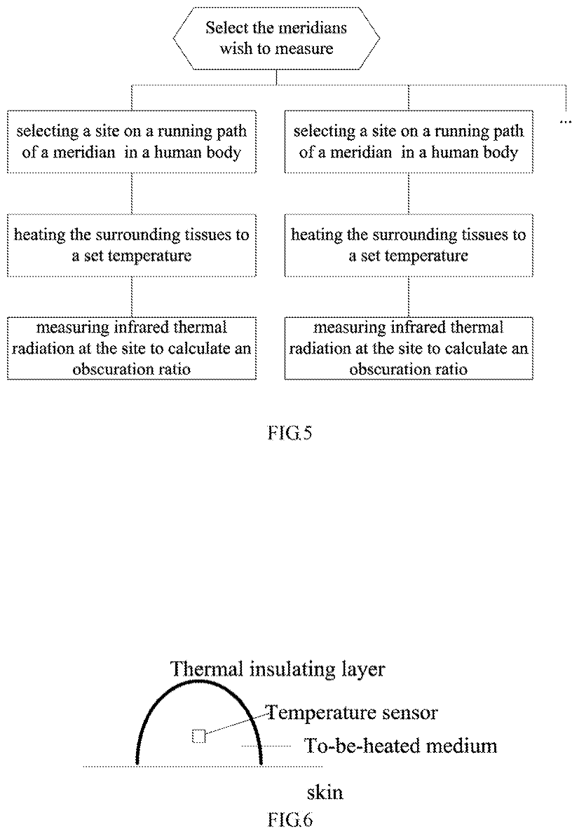

A method for measuring the strength of Wei-defensive Qi, comprising: attaching a to-be-heated medium covered with a thermal insulating layer outside to a site on a running path of a meridian in a human body, recording a temperature-rising curve of the medium, and then calculating an indicator such as a temperature-rising rate for evaluating the strength of the Wei-defensive Qi at the corresponding site. FIG. 6 is an illustration of the implementation of the method.

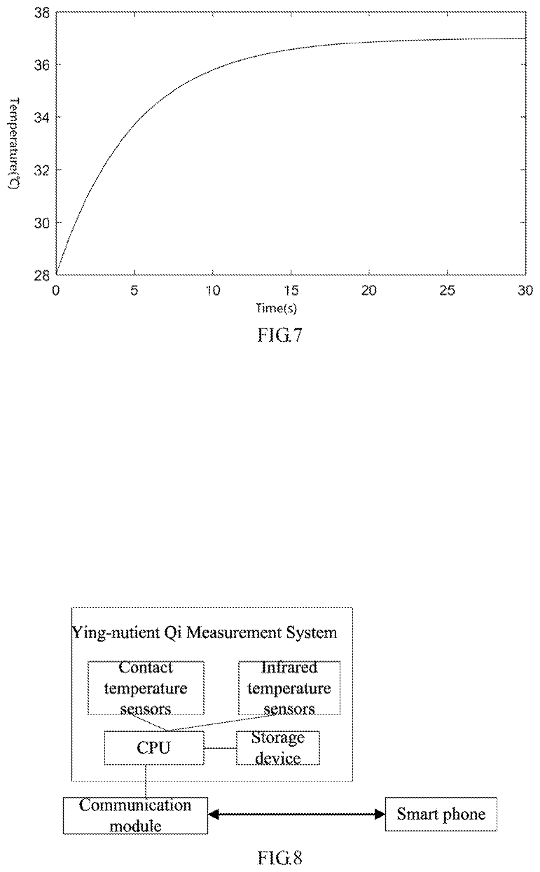

According to an embodiment of the present application, water and Resistance Temperature Detector(RTD)/thermocouple temperature sensors can be placed in a double-layer hemispherical structure with a vacuum in the middle (this structure is actually a common structure in daily life, such as a double-layer stainless steel bowl or a thermos cup). Before the test starts, use a constant temperature water tank to ensure that the water is at a certain temperature (such as 28 degrees). At the beginning of the test, the structure is covered on the tested part, and the human body will simultaneously heat the water through convection/radiation/conduction. The recorded temperature profile will resemble the charging profile of an RC circuit (FIG. 7). At this time, the strength of Wei-defensive Qi can be measured by calculating the heating rate (for example, the rising slope in the first 10 seconds). The faster the heating rate, the weaker the Wei-defensive Qi.

It should be noted that there are many other indicators to represent the strength of Wei-defensive Qi at this time. For example, assuming that the final value of the heating curve is Tf, the time to reach the temperature of 0.63 (Tf−28) (similar to RC charging time) can also be used as an indicator. The shorter the time, the weaker the Wei-defensive Qi. Of course, this is not the only indicator that can be used. Other indicators can also be calculated by using the temperature rise curve to evaluate the strength of Wei-defensive Qi.

According to an embodiment of the present application, metal can also be selected as the to-be-heated medium. At this time, different heat transfer speeds caused by different convective speeds (caused by different placement directions of the medium) will not be a problem, and the measurement results will be more accurate.

When performing the aforementioned measurements, it should also be known that Wei-defensive Qi often undergoes a sudden change at the junction of different traditional twelve two-hour periods, so the measurement should not be done during the junction time(unless one wants to know the sudden change of Wei-defensive Qi caused by the midnight-noon ebb-flow), and the measurement results must be used when the influence of measurement time is considered.

However, because a human body is a very complex organism, many factors are difficult to control precisely (such as food intake, mood, etc.). Therefore, even under strict test conditions, the heating rate obtained by this method may vary in a wide range. In other words, it is difficult to directly compare the strength of Wei-defensive Qi obtained by different people at different times. Therefore, the solution is to compare the heating rate values of different meridians of the same individual at the same time to obtain the relative strength of Wei-defensive Qi in different meridians.

According to an embodiment of the present application, furthermore: measuring the strengths of the Wei-defensive Qi in multiple meridians in a human body at the same time, and then comparing the multiple results obtained to obtain the relative strength of the Wei-defensive Qi of different meridians.

When measuring simultaneously and comparing, measuring multiple meridians at multiple fingers is convenient. Because the anatomical structures of multiple fingers are similar, the comparability is good. After obtaining the relative strength, it can be used for auxiliary diagnosis and treatment. Generally speaking, when Chinese medicine is prescribed, it is often not based on the absolute strength of Wei-defensive Qi (prolonged illness patients may have weaker Wei-defensive Qi in all meridians than healthy people) but relative strength. Note, however, that the strength of Qi & blood in any meridian should not be directly related to the name of a disease or a syndrome. The doctor must combine various other symptoms to make a judgment.

A system for measuring the strength and variation of Ying-nutrient Qi in a human body, comprising: at least two pairs of infrared temperature sensors and contact temperature sensors, a CPU, and a storage device, wherein the contact temperature sensors is in direct contact with a human body, and every infrared temperature sensor is paired with a contact temperature sensor to measure the temperature of a site.

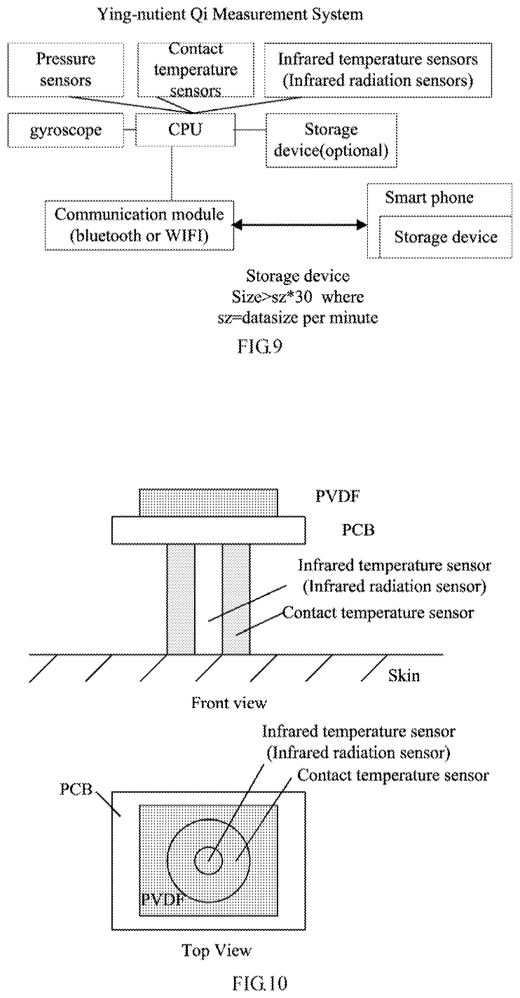

FIG. 8 is a diagram showing a structure of the system, in which a CPU, a storage device, and sensors are included. The CPU acquires the measurement results of the infrared temperature To and body surface temperature Ta from the sensors by means of communication or A/D conversion, and stores these results in the storage device. When a user makes a query, the CPU acquires the current values or historical curves of the infrared temperature and body surface temperature from the storage device, and provides them to the user in pairs simultaneously.

If a human body undergoes Qi-blood stagnation (such as disharmony between the heart and the kidney, or cold limbs) in meridians, the upstream meridians may demonstrate a significantly low infrared temperature and significantly high body surface temperature for a long time (the ΔTa=To−Ta corresponding to a certain meridian will be significantly different from other meridians). Because this is a qualitative change with relatively obvious characteristics, it can be visually observed using the present system. However, since this method requires comparison among multiple meridians, the system should provide multiple pairs of sensors.

Because the infrared temperature (due to obscuration) is lower when more Ying-nutrient Qi seeps, and the body surface temperature is higher due to heating caused by the seepage of Ying-nutrient Qi, if the strength of Wei-defensive Qi of each meridian is considered to be relative equilibrium, the difference value ΔTa=To−Ta (Note To is minuend instead of subtrahend here) between the infrared temperature To and the body surface temperature Ta may approximately represent the strength of the Ying-nutrient Qi (in particular the relative strength among multiple meridians). In general, the stronger the Ying-nutrient Qi, the smaller the corresponding ΔTa (this rule still holds in some cases where the measured infrared temperature is lower than the body surface temperature). Note that, since the body surface temperature and ΔTa are also significantly affected by the strength of the Wei-defensive Qi, and the Wei-defensive Qi often does not follow its path, this rule should be applied with care. However, coincidentally, the deficiency of Wei-defensive Qi will also reduce the body surface temperature and increase the value of ΔTa in most cases. Consequently, if the value of ΔTa in a certain meridian is significantly greater than that in other meridians, weak Ying-nutrient Qi or weak Wei-defensive Qi can be suspected. Since the thermostatic water bank and other tedious steps can be omitted, the indicator ΔTa can be used for preliminary screening or approximate judgment.

According to an embodiment of the present application, the contact temperature sensor may also be a penetrating temperature sensor (for example, a thermal resistor may be installed on the tip of an acupuncture needle).

According to an embodiment of the present application, furthermore, the infrared temperature sensors each have a waterproof structure.

There are many measures to achieve a waterproof structure. For example, the housings of sensors and electronic components can be sealed with waterproof paint or glue, or rubber seal rings, etc.

It should be noted that there is an alternative method to achieve the object of this embodiment. For example, in case the infrared temperature sensor may not have a strict waterproof design, a tester can stick the infrared sensor to a finger, then wear a waterproof glove, and immerse the hand into the thermostatic water tank. Such an alternative design is essentially the same as this embodiment, except in that the waterproof structure is expanded to include the hands of a test subject and the sensors.

Because a human body is a very complex organism in which many parameters cannot be accurately controlled, the absolute value of the strength of Ying-nutrient Qi measured by the aforementioned process can hardly be compared with a standard value, and it is more meaningful to measure the relative strength of multiple meridians. Therefore, the system should provide multiple pairs of infrared temperature sensors and contact temperature sensors simultaneously.

According to an embodiment of the present application, furthermore, the storage device stores two types of temperature data and corresponding timestamps at the same time, with a storage duration of greater than 30 minutes.

After long-term storage of temperature trends, the system may be used to monitor the abrupt changes and periodical fluctuations of the strength of the Ying-nutrient Qi. As described above, if a user has problems such as blood stasis or phlegm-fluid retention, the Qi-blood circulation will be suddenly blocked and then dredged. At this time, burrs will occur to the historical curves of the infrared temperature. Looking at the current temperature reading alone will not reveal a problem at this time, but the trend graph will provide obvious information. Furthermore, some patients (with a problem in shaoyang meridians, for example) when measured show repeatedly fluctuating infrared temperature and body surface temperature. As such, the value of infrared/body surface temperature at a certain time should not be used alone. That is to say, the trend chart over a period of time should be used here. Therefore, the storage device should be capable of storing historical information for a sufficiently long time (at least 5 minutes, and more than half an hour as recommended).

According to an embodiment of the present application, furthermore, the contact temperature sensors can be integrated in the infrared temperature sensors. Melexis's 90615 and 90632 both provide such a structure. When 90615 and 90632 are put on the skin, an object temperature To measured is the infrared temperature, and an ambient temperature Ta obtained is the body surface temperature. If 90615 is used, it should be noted that unreasonable infrared data may appear during the transition of a body surface/ambient temperature change. This is because the temperature field inside 90615 haven't become even. Therefore, these unreasonable data should be excluded with care to avoid an erroneous conclusion. Furthermore, a silicone ring with a thickness smaller than that of 90615 may also be used outside 90615, such that the sensor can be prevented from moving while ensuring the reliable contact between 90615 and the skin. Moreover, the CPU and memory of Arduino DUE may be used directly as the CPU and memory device. Here, the CPU communicates with 90615 or 90632 by using the SMBUS protocol, obtains the infrared temperature and body surface temperature simultaneously and store the same in the memory.

According to an embodiment of the present application, furthermore, the system may further include a Bluetooth or WIFI communication module for communicating with a smartphone. Since a communication function is achieved, historical records (the trend charts) in the storage device can be uploaded to the mobile phone upon the calling of software in the mobile phone. In such a way, the display/storage/analysis function of the mobile phone can be utilized to significantly prolong the storage time and reduce the volume/cost of a testing apparatus.

According to an embodiment of the present application, connecting lines (communication/power lines) may also be omitted between the CPU and the sensors. At this time, a battery and communication circuits need to be installed in the sensors. In such a way, the volume and cost of the sensors are significantly increased at present, and the recording time is reduced, which are notable disadvantages. But as time goes on and technology improves, these shortcomings may no longer be a problem,

According to an embodiment of the present application, the storage device is a storage device of a smartphone. Here, the CPU directly provides acquired data to the smartphone via communication, and the smartphone is responsible for long-term storage of two types of measured temperature curves and for displaying them to a user.

According to an embodiment of the present application, furthermore, the system includes pressure sensors for measuring the pressure of temperature sensors on the skin. FIG. 9 is an illustration of such a system. The reason for adding a pressure sensor is that although the sensor (when fixed with a tape on skin) can obtain reliable and usable data when the pressure on skin is not being monitored, if the subject inadvertently presses the temperature sensor on other objects, the temperature difference calculated from measurement can change significantly and no longer accurately reflect the strength of Ying-nutrition Qi. That is to say, the sensor's pressure on the skin affects the difference between the measured infrared temperature and the body surface temperature. To this end, it is meaningful to measure the pressure of sensors on the skin and only accept the measurement result of a temperature sensor when the pressure is less than a preset threshold (this value can be 0.098 N). Note, however, although the pressure of the temperature sensor on the skin should not be too great, the pressure should not be completely eliminated. Otherwise, the contact between the sensor and the skin is too loose, which may affect the measurement results of the contact temperature sensor.

In actual implementation, there are many options for pressure sensors, such as resistive, capacitive, piezoelectric, photoelectric, electromagnetic, Hall elements, and so on. However, considering the slight pressure and size limitations, it is more convenient to use piezoresistors or pvdf piezoelectric films. FIG. 10 shows the arrangement relationship between the pressure sensor (PVDF piezoelectric film in FIG. 10), the temperature sensor (melexis 90615 welded on a printed circuit board in FIG. 10) and the PCB(printed circuit board). Note that the pressure sensor is not installed between the temperature sensor and the skin but on the other side of the temperature sensor(away from the skin). This is because placing the pressure sensor (thin film) between the temperature sensor and the skin introduces thermal resistance and blocks infrared light, thereby interfering with the temperature sensor's operation. In order to avoid this problem, it is logical to place the pressure sensor on the side away from the skin of the temperature sensor and then use the transmissibility of force to measure.

Since the pressure sensor often does not provide a communication interface, a charge amplifier and A/D conversion (for pvdf) or other signal conditioning circuits (for other pressure sensors such as piezoresistive or capacitive) is needed in practical applications so that the CPU can get the pressure value. Since the implementation method is well known to those skilled in the art, details are not provided here.

When measuring, the two temperature sensors (or their combined structure) should generally be in contact with the skin with slight pressure. Therefore, the measuring range of the pressure sensor should be 0-100 g force (corresponding to 0-0.98 N). For accurate measurement, the measurement range can be further narrowed to 50 g or 20 g force, and an over-range (over pressure) alarm is provided at the same time.

It should also be noted that although such measurement systems typically collect signals on fingers, it is also possible to place sensors on other meridian-passing body parts (such as the face). At this time, the composite structure of the temperature sensor and the pressure sensor can be pasted on the skin with a tape (the tape should apply pressure to the temperature sensor indirectly through PVDF). Under normal circumstances, the value is usable when the reading of the pressure sensor is below 10 g force (0.098 N). Otherwise, the clamping or sticking force on the sensor should be adjusted to reduce the pressure to a reasonable value.

According to an embodiment of the present application, furthermore, the system includes a mechanism capable of precisely controlling the pressure of the temperature sensors on the skin. The reason lies that in some occasions, it may be useful to precisely control the pressure of temperature sensor on the skin, so that more accurate results can be obtained. FIG. 11 gives a front view of the corresponding structure (looking from the fingertip to the palm). FIG. 11 uses a base that spans the fingers. The actuator(the push rod motor is taken as an example in FIG. 11) and the pressure sensor, temperature sensor, etc., are all attached to this base (But the temperature sensor cannot be rigidly fixed on the base. Otherwise, the pressure sensor cannot measure the pressure value). In the implementation, it is also possible to form a closed-loop control of “measure the pressure—the pressure is compared with an expected pressure—issue instructions to the actuator to correct the clamping force” to fix the pressure at a desired value (Note that this value need to be small enough. Otherwise the temperature difference ΔTa will be unusable). Obviously, any actuator that can expand and squeeze the temperature sensor can be used here. It is not possible to exhaustively enumerate all possible actuator that can be used. However, common implementation means can be given: for example, the repulsive force between two electromagnets can be used, or the repulsive force between a permanent magnet and an electromagnet can be used, or a hydraulic (or pneumatic) driven piston can be used, Alternatively, linear motors, push rod motors, etc. may also be used. These methods have corresponding advantages and disadvantages. For example, electromagnets will generate heat, affecting the temperature sensor's measurement results. The hydraulic (or pneumatic) pistons will be easily damaged, while using push rod motors will be more expensive. In actual implementation, the pros and cons need to be weighed to make a choice.

According to an embodiment of the present application, furthermore, the system includes one or more gyroscopes for detecting subject motion. FIG. 9 is an illustration of such a system. The gyroscope is needed because if the subject (person measured) remains still during the measurement, the difference between the infrared and body surface temperatures can accurately represent the strength of Ying-nutrition Qi. However, suppose the subject changes posture during the long-term measurement process. In that case, the blood flow rate under the skin may change, resulting in rapid and significant changes in infrared and body surface temperature. Therefore, in the analysis, it is meaningful to exclude the data obtained during these transitions. Although the time when the subject changes posture can be recorded by the tester or the subject himself, it is more convenient to add one or more gyroscopes to the measurement system and stick the gyroscopes on the subject during the test. Then the CPU can communicate with the gyroscope, obtains the speed and acceleration information of the subject while obtaining the temperature data, and stores these information together with the temperature data. The gyroscope and temperature data can be displayed together when the data is finally analyzed. If the acceleration or speed exceeds a certain threshold(the threshold can be 0.1 m/s) the data for a certain period of time (such as 5 minutes) should not be used. Note the threshold is not necessary here. Other means like fuzzy logic or artificial intelligence can also be used to exclude data without need of a threshold. The number of gyroscopes used needs to be determined by the distribution of the sensors. For example, assuming all temperature sensors are on the fingers of one hand, a single gyroscope could be taped to the palm or back of the hand. If the temperature sensors are distributed on both hands, two gyroscopes need to be pasted on both hands, and so on.

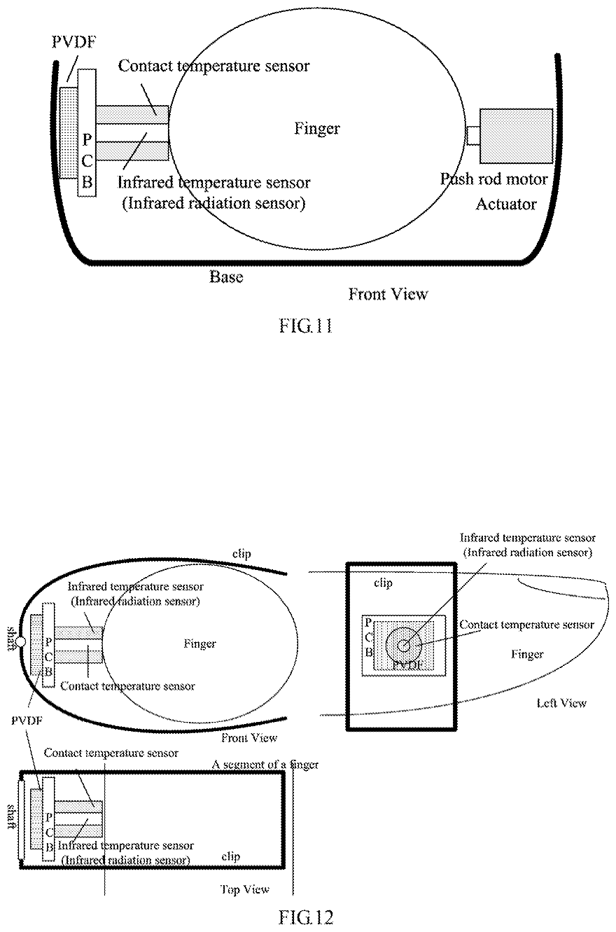

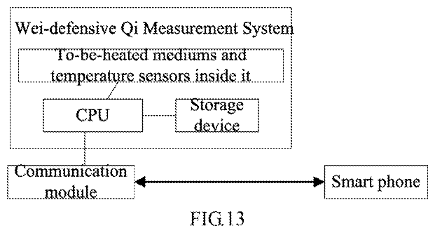

According to an embodiment of the present application, furthermore, the system includes clips capable of fixing the temperature sensors on a side of fingers. Note the clips can be separate components. And the clamping force direction of the clip should be different from the direction in which the temperature sensor is located. FIG. 12 shows a schematic diagram of the corresponding sensor arrangement (but there are additional pressure sensors in the figure). This kind of clip can use a structure in which two parts are combined through a rotating shaft (with a spring inside like a clothes clip), or it can be an integrated structure without a shaft. If a shaft is not used, the clip can be made of silicone rubber or other resilient metal that tends to regain shape (for example, the metal that makes springs or elastic watch straps can be used). It can be seen that when the clip is trying to restore its shape, the clamping force provided is in the up and down direction of the finger (here, the back of the hand is defined as the upper direction and palm, the lower direction), which is different from the direction of the temperature sensor. When in use, if the clamping position is closer to the far side (the side away from the temperature sensor), the pressure on the sensor will be greater; if the clamping position is closer to the near side(the side close to the sensor), the pressure on the sensor will be smaller. By adjusting the clamping position, it is possible to avoid excessive pressure on the skin while ensuring reliable contact of the temperature sensor with the skin. The reason for choosing the clamping direction (up and down direction of the fingers) is that the human meridians on the hand are generally distributed on the sides of fingers. Consequently, in order to avoid blocking the flow of Qi when fixing the temperature and pressure sensors, one should avoid applying pressure to the sides of fingers. In contrast, it is much safer to apply pressure in the up and down direction of a finger. This has also been verified in practice for a long time (the clip of blood oxygen monitoring probes generally applies pressure in the up and down direction).

According to an embodiment of the present application, furthermore, the system includes pressure sensors for measuring the pressure of the temperature sensors on the skin. FIG. 12 shows the specific implementation's side view, top view, and front view (from the fingertip to the palm). This approach further includes pressure sensors for measuring the pressure of temperature sensors on the skin (the implementation method and measurement range of the pressure sensors have been mentioned above). Excessive pressure on temperature sensors can be avoided by reading the pressure reading and adjusting the clamping position (But slight pressure is still needed to ensure temperature sensor and skin contact). Note that the printed circuit board and temperature sensor should not be rigidly fixed on the clip. Otherwise, pressure sensor cannot use the transmissibility of force to measure the pressure of temperature sensors on the skin.

It should also be noted that infrared radiation sensors can replace the infrared temperature sensors mentioned in previous implementations. This is because no matter the infrared temperature sensor uses a thermopile or a photoelectric device, it measures the intensity of the infrared radiation and establishes a relationship between the intensity of the infrared radiation and the measured temperature (according to Planck's black body radiation law). After the seepage of Ying-nutrient Qi shields the infrared rays, the measured infrared radiation no longer reflects a skin (or underneath tissue) temperature, and the infrared spectrum also deviates from the black body radiation spectrum. However, the temperature calculated according to the measured infrared radiation intensity still indirectly reflects the radiation intensity. Therefore, temperature difference used to measure the strength of Ying-nutrition Qi can be replaced with difference between infrared radiation Ro and a virtual radiation Ra, which corresponds to the body surface temperature Ta. Note that the temperature corresponding to Ro is higher than the body surface temperature Ta, so Ra is generally smaller than Ro, but this does not prevent Ra from being used as a reference to measure the degree of infrared obscuration(the smaller the Ro-Ra, the stronger the strength of Ying-nutrient Qi). According to this principle, several other implementations in which the infrared radiation sensor (instead of the infrared temperature sensor) is used can be obtained:

A system for measuring the strength and variation of Ying-nutrient Qi in a human body, comprising: at least two pairs of infrared radiation sensors and contact temperature sensors, a CPU, and a storage device, wherein the contact temperature sensors are in direct contact with a human body, and every infrared radiation sensor is paired with a contact temperature sensor to measure a site.

FIG. 9 is a diagram showing an overall structure of the system, in which a CPU, a storage device, and sensors are included. The CPU acquires the measurement results of the infrared radiation Ro and body surface temperature Ta from the sensors by means of communication or A/D conversion, and stores these results in the storage device. When a user makes a query, the CPU acquires the current values or historical curves of the infrared radiation and body surface temperature from the storage device, and provides them (or values derived from them) to the user in pairs simultaneously.

Because a human body is a very complex organism in which many parameters cannot be accurately controlled, the absolute value of the strength of Ying-nutrient Qi measured by the aforementioned process can hardly be compared with a standard value, and it is more meaningful to measure the relative strength of multiple meridians at the same time. Therefore, the system should provide multiple pairs of infrared radiation sensors and contact temperature sensors simultaneously.

According to an embodiment of the present application, furthermore, the storage device stores two types of data and corresponding timestamps at the same time, with a storage duration of greater than 30 minutes.

According to an embodiment of the present application, furthermore, the system also includes a Bluetooth or WIFI communication module for communicating with a smartphone. Since a communication function is achieved, historical records (the trend charts) in the storage device can be uploaded to a mobile phone upon the calling of software in the mobile phone. In such a way, the display/storage/analysis function of the mobile phone can be utilized to significantly prolong the storage time and reduce the volume/cost of a testing apparatus.

According to an embodiment of the present application, the storage device is a storage device of a smartphone. Here, the CPU directly provides acquired data to the smartphone via communication, and the smartphone is responsible for long-term storage of two types of data and for displaying them to a user.

According to an embodiment of the present application, furthermore, the system also includes pressure sensors for measuring the pressure of temperature sensors on the skin. By using a pressure sensor, the incorrect measurement can be discarded when the temperature sensor is under too much pressure. In actual implementation, only one pressure sensor is needed for a contact temperature sensor if the infrared radiation sensor and the contact temperature sensor are integrated or the infrared radiation sensor is not in contact with the skin. Otherwise, two pressure sensors are needed. The possible forms and installation position of the pressure sensor are mentioned above. In addition, because the pressure sensor often does not provide a communication interface, a supporting charge amplifier and A/D conversion (for PVDF) or other signal conditioning circuits (for other forms of pressure sensors such as piezoresistive or capacitive) is needed in practical applications so that the CPU can get the pressure value. However, the specific implementation method is well known to those skilled in the art, so details are not provided here.

According to an embodiment of the present application, further, the system includes one or more gyroscopes for detecting subject motion. Then the CPU uses a communication method (such as IIC or SMBUS, etc.) to communicate with the gyroscope, obtains the speed and acceleration information of the subject while obtaining the radiation/temperature data, and stores this information together with the radiation/temperature data. The gyroscope and radiation/temperature data can be displayed together when the data is finally analyzed. If the acceleration or speed exceeds a certain threshold(the threshold can be 0.1 m/s), the data for a certain period of time (such as 5 minutes) should not be used. Note the threshold is not necessary here. Other means like fuzzy logic or artificial intelligence can also be used to exclude data without need of a threshold.

According to an embodiment of the present application, furthermore, the system includes clips capable of fixing the temperature sensors on a side of fingers. Note the clamping force direction of the clip should be different from the direction in which the temperature sensor is located. FIG. 12 shows a schematic diagram of the corresponding sensor arrangement (but there are additional pressure sensors in the figure). This kind of clip can use a structure in which two parts are combined through a rotating shaft (with a spring inside like a clothes clip), or it can be an integrated structure without a shaft. If a shaft is not used, the clip can be made of silicone rubber or other resilient metal that tends to regain shape (for example, the metal that makes springs or elastic watch straps can be used). It can be seen that when the clip is trying to restore its shape, the clamping force provided is in the up and down direction of the finger (here, the back of the hand is defined as the upper direction and palm, the lower direction), which is different from the direction of the temperature sensor. When in use, if the clamping position is closer to the far side (the side away from the temperature sensor), the pressure on the sensor will be greater; if the clamping position is closer to the near side (the side close to the sensor), the pressure on the sensor will be smaller. By adjusting the clamping position, it is possible to avoid excessive pressure on the skin while ensuring reliable contact of the temperature sensor with the skin.

A system for measuring the strength of Wei-defensive Qi, comprising: a CPU, a storage device, at least two to-be-heated mediums capable of contacting and being heated by a human body, and corresponding temperature sensors placed within the mediums, wherein the CPU records a temperature-rising curve of the to-be-heated medium, and calculates an indicator such as a temperature-rising rate for evaluating the strength of the Wei-defensive Qi at the corresponding site. FIG. 13 shows such a system. FIG. 6 is a detailed structural diagram of the to-be-heated medium, temperature sensor, and thermal insulating layer.

According to an embodiment of the present application, the to-be-heated medium may be a small amount of water (or a flowable medium such as alcohol), and a thermal resistance/thermocouple temperature sensor is placed in the water in the structure. Before the test starts, use a constant temperature water tank to ensure that the water is at a specific temperature (such as 28 degrees). At the beginning of the test, the structure is put upon the tested site, and the human body will simultaneously heat the water through convection/radiation/conduction. The recorded temperature curve will be similar to the charging curve of an RC circuit. At this time, the strength of Wei-defensive Qi can be measured by calculating the heating rate (for example, the rising slope in the first 10 seconds). The faster the heating rate, the weaker the Wei-defensive Qi.

It should be noted that there are many other indicators to represent the strength of Wei-defensive Qi at this time. For example, assuming that the final value of the heating curve is Tf, the time to reach the temperature of 0.63 (Tf−28) (similar to RC charging time) can also be used as an indicator. The shorter the time, the weaker the Wei-defensive Qi. Of course, this is not the only indicator that can be used. Other indicators can also be calculated by using the temperature rise curve data to evaluate the strength of Wei-defensive Qi.

According to an embodiment of the present application, the to-be-heated medium can also be metal (such as aluminum, copper, tin, stainless steel or hybrid structure) or other immobile substances, and the thermal resistance/thermocouple temperature sensor is placed in the middle of the to-be-heated medium. The advantage of this method is that the to-be-heated medium does not have internal convection, so the measurement results will not be affected by changes in the heat transfer rate (from the human body to the medium) caused by the sensor placement direction.

According to an embodiment of the present application, furthermore, the to-be-heated medium is covered with a thermal insulating layer.

The thermal insulating layer can use a double-layer hemispherical structure with a vacuum in the middle (this structure is a common structure in daily life, such as double-layer stainless steel bowls or thermos cups). Measurements are more accurate when the to-be-heated medium is covered with thermal insulation.

Since a human body is a very complex organism, many parameters cannot be precisely controlled, it may be difficult to compare the value of the strength of the Wei-defensive Qi obtained by the aforementioned method with a standard value. It is more meaningful to measure the relative strength of different meridian Wei-defensive Qi at the same time. Therefore, the system should provide multiple sets of to-be-heated mediums and temperature sensors at the same time.

According to an embodiment of the present application, furthermore, the system may include a Bluetooth or WIFI communication module for communicating with a smartphone. Since a communication function is achieved, historical records (the trend charts) in the storage device can be uploaded to a mobile phone upon the calling of software in the mobile phone. In such a way, the display/storage/analysis function of the mobile phone can be utilized to significantly prolong the storage time and reduce the volume/cost of a testing apparatus.

According to an embodiment of the present application, connecting lines (communication/power lines) may also be omitted between the CPU and the sensors. Here, a battery and communication circuits need to be installed in the sensors. In such a way, the volume and cost of the sensors are significantly increased at present, and the recording time is reduced, which are notable disadvantages. But as time goes on and technology improves, these shortcomings may no longer be a problem,

According to an embodiment of the present application, the storage device may also be a storage device of a smartphone. Here, the CPU directly provides acquired data to the smartphone via communication, and the smartphone is responsible for long-term storage of two types of measured data and for displaying them to a user.

There are many situations in which the method and system of the present application can be used. For example, when it is suspected that the patient has blood stasis or phlegm-fluid retention, the strength of the Ying-nutrient Qi can be continuously monitored.

When the ambient temperature is precisely controlled, the Ying-nutrient Qi (infrared temperature) in the meridians of healthy people is stable and changes little over time within an hour.

If the corresponding infrared temperature has glitches, as shown in FIG. 2, it can be suspected that the patient has blood stasis or phlegm-fluid retention in the meridians (FIG. 2 shows the observation results on the middle section of the little finger of Hand Shaoyin meridian between 5 am-7 am after the blood stasis happen on the Foot Yangming meridian. Note that because the strength of Qi becomes weaker after 7 am, the burr disappears immediately after 7 am).

However, it should be noted that one must not drink alcohol before the test (otherwise, there will be a similar curve), and even if there is blood stasis/phlegm-fluid retention, it is not always possible to observe glitches everywhere. Because according to midnight-noon ebb-flow (the sequence of Qi flow in meridians), the strength of Qi in different meridians alternately decline and prosper in different traditional twelve two-hour periods. Most of the time, if the place blocked is downstream of the meridian where Qi & blood are flourishing, the clear results can be seen by observing the meridians near the blocked point.

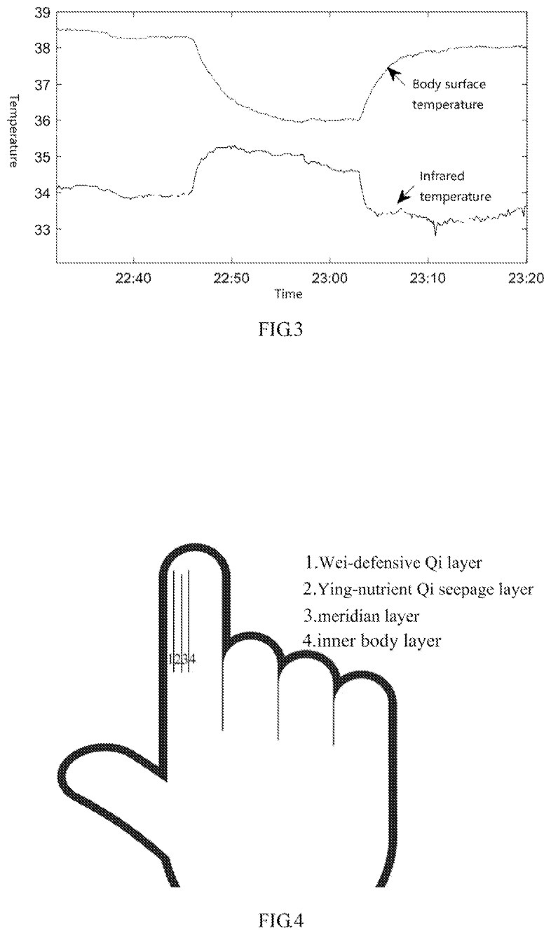

In addition, it should also be pointed out that if the blockage of a meridian is so serious that it is completely blocked, the burr cannot be seen. But if measured at the right time according to the Midnight-noon ebb-flow, it will be observed that the contact temperature (body surface temperature) on some upstream points of the blockage point rises at certain moments while the infrared temperature falls (and vice versa on downstream points). This is because the heat generated by the oozing of Ying-nutrient gas increases significantly. Hence, the internal layer and body surface temperature rise, but the shading coefficient also increases, so the infrared temperature drops. FIG. 3 shows such a measured waveform. The meridian blockage cannot be judged by the infrared temperature change alone. However, if the temperature obtained by the contact temperature sensor is also used, the probability of blockage will be very high when the two change in reverse.

In addition to blood stasis/phlegm retention that can lead to blockage of meridians, other reasons can also cause Qi & blood blockage (such as disharmony between the heart and the kidney or cold limbs). At this time, the changing waveform shown in FIG. 3 may not appear, but the places where upstream meridians run may have a noticeably low infrared temperature and noticeably high body surface temperature for a long time. Therefore, when measuring multiple meridians (especially when measuring on multiple fingers) simultaneously, the manifestation of abnormal meridians will be noticeably different from other meridians, and it is not difficult to make a judgment in conjunction with other symptoms at this time.

Of course, according to traditional Chinese medicine theory, blood stasis or phlegm-fluid retention (or meridian blockage) does not directly correspond to any specific disease (for example, blood stasis may cause insomnia, coronary heart disease, tumor, and many other diseases, but other reasons may also be the cause of these diseases). Moreover, it does not correspond to any specific prescription or treatment method (for example, sometimes Xuefu Zhuyu Decoction should be used to cure blood stasis, but sometimes Guizhi Fuling Pills should be used). Therefore, although the method and system of the present application can give valuable hints, it is neither a diagnosis method nor a treatment method for diseases.

In addition to judging the blockage of meridians, the method and system of the present application can also be used to assist pattern differentiation by the six meridians.

Pattern differentiation of the six meridians is a diagnosis and treatment system proposed by medical sage Zhang Zhongjing. Using the six meridians to differentiate syndromes can get better result with less effort. The corresponding classical formulas prescribed are often effective soon after the patient takes medicine.

However, the current method classifies a patient into a certain syndrome in accordance with the clustering of multiple symptoms. For example, sweating more may be a sign of Taiyang Meridian disease or Yangming Meridian disease, but combined with a floating pulse, there is a high probability that the patient gets Taiyang Meridian disease; if combined with thirst, there is a high probability that the patient gets Yangming Meridian disease.

In other words, the relationship between symptoms and six meridian diseases is not one-to-one but a many-to-many relationship. There cannot be decoupling between symptoms and disease pattern differentiation (not a specific disease but six disease pattern differentiation such as Taiyang disease and Shaoyang disease). Although “Treatise on the Six Seasonal Phases and the Visceral Picture” states that “For pulse strength in carotid: grade 1 means disease in Shaoyang meridian, grade 2 means disease in the Taiayng meridian, grade 3 in Yangming meridian, and grade 4 or more diseases means excessive yin rejecting yang. For pulse strength in the Cunkou area: grade 1 means disease in the Jueyin meridian, grade 2 means disease in the Shaoyin meridian, grade 3 means disease in the Taiyin meridian, grade 4 means blocked yin”, but it is challenging to apply.

Using the method and system of the present application, one can intuitively see the strength and change of the Ying-nutrient Qi/Wei-defensive Qi of the six meridians, and this can achieve decoupling. That is, it can help the doctors using the classic formulas to classify the diseases into one of the pattern differentiation of six meridians (or its combination). For example, under the premise of precisely controlling the ambient temperature, if a doctor sees constant fluctuations of the strength of Ying-nutrient Qi (infrared temperature) in a patient's Hand Shaoyang meridian, he can guess that it is a Shaoyang meridian disease. Because the Shaoyang meridian is a hub, Qi & blood will flow back and forth between the Taiyang meridian and Yangming meridian continuously. Similarly, if a doctor sees that Ying-nutrient Qi/Wei-defensive Qi in Yangming meridian is very strong, namely, obviously stronger than other meridians, he can guess it is a Yangming meridian disease. This approach can provide specific auxiliary information in addition to a large number of uncoupled information, so it has a unique value.

When assisting diagnose the pattern differentiation by the six meridians, it should also be noted that a human body's Ying-nutrient Qi and Wei-nutrient Qi fluctuate continuously throughout the day (but are nearly stable within an hour). If a doctor wants to determine the pattern differentiation by the six meridians, he should also consider the time when the disease is about to be relieved (and is about to worsen), which is described in the ShangHanLun, and measure at the appropriate time. He should not expect clear results if the patient is measured at some random time.

In addition, it should be noted that the pattern differentiation by the six meridians obtained (by using the method and the system of the present application) does not directly correspond to any specific disease, nor does it correspond to any specific prescription or treatment method. Therefore, although the method and system of the present application can give valuable hints, it is neither a diagnosis method nor a treatment method for diseases.

What is claimed is:

1. A method for measuring the strength of Ying-nutrient Qi, comprising:
selecting a site on a running path of a meridian in a human body, heating the surrounding tissues to a set temperature, and then measuring infrared thermal radiation at the site to calculate an obscuration ratio, thereby obtaining a numeralized representation of the strength of the Ying-nutrient Qi.

2. The method according to claim 1, further comprising: measuring the strengths of the Ying-nutrient Qi in multiple meridians in a human body at the same time, and then comparing the multiple results obtained to obtain the relative strength of the Ying-nutrient Qi among different meridians.

3. A system for measuring the strength and variation of Ying-nutrient Qi in a human body, comprising: at least two pairs of infrared temperature sensors and contact temperature sensors, a CPU, and a storage device, wherein the contact temperature sensors is in direct contact with a human body, and every infrared temperature sensor is paired with a contact temperature sensor to measure the temperature of a site.

4. The system according to claim 3, wherein the storage device stores two types of temperature data and corresponding timestamps at the same time, with a storage duration of greater than 30 minutes.

5. The system according to claim 3, further comprising: a Bluetooth or WIFI communication module for communicating with a smartphone.

6. The system according to claim 3, wherein the storage device is a storage device of a smartphone.

7. The system according to claim 3, further comprising: pressure sensors for measuring the pressure of temperature sensors on the skin.

8. The system according to claim 7, further comprising: a mechanism capable of precisely controlling the pressure of the temperature sensors on the skin.

9. The system according to claim 7, further comprising: one or more gyroscopes for detecting subject motion.

10. The system according to claim 3, further comprising: clips capable of fixing the temperature sensors on a side of fingers.

11. The system according to claim 10, further comprising: pressure sensors for measuring the pressure of the temperature sensors on the skin.

12. A system for measuring the strength and variation of Ying-nutrient Qi in a human body, comprising: at least two pairs of infrared radiation sensors and contact temperature sensors, a CPU, and a storage device, wherein the contact temperature sensors are in direct contact with a human body, and every infrared radiation sensor is paired with a contact temperature sensor to measure a site.

13. The system according to claim 12, wherein the storage device stores two types of data and corresponding timestamps at the same time, with a storage duration of greater than 30 minutes.

14. The system according to claim 12, further comprising: a Bluetooth or WIFI communication module for communicating with a smartphone.

15. The system according to claim 12, wherein the storage device is a storage device of a smartphone.

16. The system according to claim 12, further comprising: pressure sensors for measuring the pressure of temperature sensors on the skin.

17. The system according to claim 16, further comprising: one or more gyroscopes for detecting subject motion.

18. The system according to claim 12, further comprising: clips capable of fixing the temperature sensors on a side of fingers.

19. A system for measuring the strength of Wei-defensive Qi, comprising: a CPU, a storage device, at least two to-be-heated mediums capable of contacting and being heated by a human body, and corresponding temperature sensors placed within the mediums, wherein the CPU records a temperature-rising curve of the to-be-heated medium, and calculates an indicator such as a temperature-rising rate for evaluating the strength of the Wei-defensive Qi at the corresponding site.

20. The system according to claim 19, wherein the to-be-heated medium is covered with a thermal insulating layer.
